(19) United States
(12) Patent Application Publication
(10) Pub. No.: US 2004/0260354 A1
(43) Pub. Date: Dec. 23, 2004

(54) Miniature compression feedthrough assembly for electrochemical devices

(76) Inventors: Christian S. Nielsen, River Falls, WI (US); Timothy T. Bomstad, Inver Grove Heights, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/463,750

(22) Filed: Jun. 17, 2003

(51) Int. Cl. A61N 1/375

(52) U.S. Cl. 607/37

(57) ABSTRACT

A miniature insulative feedthrough receives an electrical lead therethrough and includes a ferrule having first and second open ends and an interior surface. At least a first insulating ring is positioned within the ferrule and has an aperture therethrough for receiving the electrical lead. At least one compression ring is positioned within the ferrule for sealingly engaging the interior surface, the compression ring also having an aperture therethrough for receiving the electrical lead. First and second retaining portions are provided for maintaining the insulating ring and the compression ring in position within the ferrule.

### FIELD OF THE INVENTION

The present invention relates generally to electrochemical components, and more particularly to a miniature compression feedthrough assembly for use in electrolytic capacitors, batteries and the like utilized in conjunction with implantable medical devices.

### BACKGROUND OF THE INVENTION

The trend toward reductions in the size and thickness of implantable medical devices such as implantable cardioverter-defibrillators (ICDs) has led to the need for miniaturization of the electrochemical components utilized in such devices. Capacitors, for example, are employed in ICDs typically implanted in a patient's chest to treat very fast, and potentially lethal, cardiac arrhythmias. These devices continuously monitor the heart's electrical signals and sense if, for instance, the heart is beating dangerously fast. If this condition is detected, the ICD can deliver one or more electric shocks, within about five to ten seconds, to return the heart to a normal heart rhythm. These electrical stimuli may range from a few micro-joules to very powerful shocks of approximately twenty-five joules to forty joules.

Early generations of ICDs utilized high-voltage, cylindrical capacitors to generate and deliver defibrillation shocks. For example, standard wet slug tantalum capacitors generally have a cylindrically shaped conductive casing serving as the terminal for the cathode and a tantalum anode connected to a terminal lead electrically insulated from the casing. The opposite end of the casing is also typically provided with an insulator structure. One such capacitor includes a metal container that functions as a cathode. A porous coating, including an oxide of a metal selected from a group consisting of ruthenium, iridium, nickel, rhodium, platinum, palladium, and osmium, is disposed proximate an inside surface of the container and is in electrical communication therewith. A central anode selected from the group consisting of tantalum, aluminum, niobium, zirconium, and titanium is spaced from the porous coating, and an electrolyte within the container contacts the porous coating and the anode.

While the performance of these capacitors was acceptable for defibrillator applications, efforts to optimize the mechanical characteristics of the device have been limited by the constraints imposed by the cylindrical design. In an effort to overcome this, flat electrolytic capacitors were developed. One such capacitor comprises a deep-drawn sealed capacitor having a generally flat, planar geometry. The capacitor includes at least one electrode provided by a metallic substrate in contact with a capacitive material. The coated substrate may be deposited on a casing side-wall or connected to a side-wall. The capacitor has a flat planar shape and utilizes a deep-drawn casing comprised of spaced apart side-walls Joined at their periphery by a surrounding intermediate wall. Cathode material is typically deposited on an interior side-wall of the conductive encasement which serves as the negative terminal for the electrolytic capacitor, though such material may also be deposited on a separate substrate and electrically coupled to the capacitor encasement. The other capacitor terminal (i.e. the anode) is isolated from the encasement by an insulator or feedthrough structure including, for example, a glass-to-metal seal. In accordance with one known technique, an anode lead (e.g. tantalum) imbedded into the anode is laser welded to a terminal lead that passes through the ferrule. This anode lead-to-feedthrough terminal weld joint (i.e. cross-wire weld) is formed by shaping one or more of the leads into a “U” or “J” shape, pressing the terminal ends of the leads together, and laser welding the interface.

A valve metal anode made from metal powder is pressed and sintered to form a porous structure, and a wire (e.g. tantalum) is imbedded into the anode during pressing to provide a terminal for joining to the feedthrough. A separator (e.g. polyolefin, a fluoropolymer, a laminated film, non-woven glass, glass fiber, porous, ceramic, etc.) is provided between the anode and the cathode to prevent short circuits between the electrodes. Separator sheets are sealed either to a polymer ring that extends around the perimeter of the anode or to themselves.

A separate weld ring and polymer insulator may be utilized for thermal beam protection as well as anode immobilization. Prior to encasement welding, a separator encased anode is joined to the feedthrough wire by, for example, laser welding. This joint is internal to the capacitor. The outer metal encasement structure is comprised essentially of two symmetrical half shells that overlap and are welded at their perimeter seam to form a hermetic seal. This weld is referred to as a rotary weld since the part is welded as it rotates on its side rather than employing a top-down approach. Alternatively, a top-down approach may be utilized to weld a lid onto a deep-drawn container. After welding, the capacitor is filled with electrolyte through a port in the encasement.

The above described techniques present concerns relating to both device size and manufacturing complexity. The use of overlapping half-shields results in a doubling of the encasement thickness around the perimeter of the capacitor thus reducing the available interior space for the capacitor's anode. This results in larger capacitors. Space for the anode material is further reduced by the presence of the weld ring and space insulator. In addition, manufacturing processes become more complex and therefore more costly, especially in the case of a deep-drawn encasement.

The abovementioned method of joining an anode lead to a terminal lead was found to be problematic, however, as the step of cross-wire welding must be performed prior to welding the feedthrough ferrule to the capacitor encasement or sufficient space must be provided in the capacitor anode structure to facilitate clamping and welding following ferrule welding. Producing the cross-wire weld prior to ferrule welding subjects the materials employed in the feedthrough seal to thermal stress and increases the cost and complexity of manufacture. Conversely, performing cross-wire welding after ferrule welding has a negative impact on volumetric efficiency.

As mentioned above, it is common for the anode terminal to be isolated from the encasement by an insulator or feedthrough structure comprised including a glass-to-metal seal. Such seals are well known in the art. To avoid problems which may be encountered due to the rigidity of glass-to-metal seals, polymer-to-metal seals have been employed. For example, it is known to secure an anode lead within a ferrule by means of a series of polymeric sealing layers. These layers may comprise a first layer of a synthetic polymeric material forming a plug on end of the ferrule internal to the electrolytic cell, a second layer of synthetic polymeric material disposed within the ferrule, and a third layer of glass disposed within the ferrule to provide a hermetic seal. Similar assemblies, varying in the arrangement and/or shape of the polymeric layers, are also known. Unfortunately, current methods of manufacturing such assemblies are relatively complex, time-consuming, and expensive.

It should thus be appreciated that it would be desirable to provide an electrochemical device including an improved feedthrough assembly that is volumetrically efficient and simple to manufacture.

### BRIEF SUMMARY OF THE INVENTION

According to a broad aspect of the invention there is provided a miniature insulative feedthrough for receiving an electrical lead therethrough. The feedthrough includes a ferrule having first and second open ends and an interior surface. At least a first insulating ring is positioned within the ferrule and has an aperture therethrough for receiving the electrical lead. At least one compression ring is positioned within the ferrule for sealingly engaging the interior surface, the compression ring also having an aperture therethrough for receiving the electrical lead. First and second retaining portions are provided for maintaining the insulating ring and the compression ring in position within the ferrule.

According to a further aspect of the invention there is provided a method for feeding a terminal lead through an encasement wall of an electrochemical cell of the type utilized in implantable medical devices to the exterior of the electrochemical cell. A ferrule having first and second ends is positioned through the encasement wall, the ferrule having an interior surface and a crimping region at the first end. The terminal lead is passed through the ferrule. The terminal lead is then threaded through an assembly comprised of at least first and second insulating rings and at least one intermediate compression rings. The assembly is then positioned within the ferrule, and the crimping portion and the assembly are compressed to maintain the assembly within the ferrule and to deform the compression ring so as to sealingly engage the interior surface.

According to a still further aspect of the invention there is provided an electrochemical cell for use in an implantable medical device. The electrochemical cell comprises a shallow drawn encasement having at least one electrode body disposed within the encasement. An electrical lead is coupled to the body, and an insulative feedthrough is coupled through the encasement for receiving the electrical lead therethrough. The insulative feedthrough comprises a ferrule having first and second open ends and an interior surface. At least first and second insulating rings are positioned within the ferrule each having an aperture therethrough for receiving the electrical lead. At least one compression ring is positioned within the ferrule and compressed between the first and second insulating rings so as to sealingly engage the interior surface, the compression ring also having an aperture therethrough for receiving the electrical lead. First and second retaining portions are provided for maintaining the first and second insulating rings in position within the ferrule.

### BRIEF DESCRIPTION OF THE DRAWINGS

The present invention will hereinafter be described in conjunction with the following drawing figures, wherein like numerals denote like elements, and

FIG. 1 is a cross-sectional view of an electrolytic capacitor in accordance with the teachings of the prior art;

FIGS. 2, 3, and 4 are front, side, and top cross-sectional views of a flat electrolytic capacitor in accordance with the teachings of the prior art;

FIGS. 5, 6, and 7 are front cross-sectional, side cross-sectional, and scaled cross-sectional views of a novel electrolytic capacitor;

FIG. 8 is a cross-sectional view of a capacitor/anode encasement structure in accordance with the teachings of the prior art;

FIG. 9 is a cross-sectional view of a novel capacitor/anode encasement assembly;

FIG. 10 is a cross-sectional view of an alternative capacitor/anode encasement assembly;

FIGS. 11 and 12 are cross-sectional and cutaway isometric views of a feedthrough assembly in accordance with the first embodiment of the present invention;

FIGS. 13, 14, and 15 are front, bottom, and isometric views of a ferrule of the type utilized in conjunction with the inventive feedthrough assembly shown in FIGS. 11 and 12;

FIGS. 16 and 17 are top and cross-sectional views of an insulating ring suitable for use in conjunction with the feedthrough assembly illustrated in FIGS. 11 and 12;

FIGS. 18 and 19 are isometric and cross-sectional views of a compression ring suitable for use in the inventive feedthrough assembly shown in FIGS. 11 and 12;

FIG. 20 is a cross-sectional view of the feedthrough assembly shown in FIGS. 11 and 12 in a compressed state;

FIG. 21 is a front view of a second embodiment of a ferrule suitable for use in conjunction with the inventive feedthrough assembly;

FIG. 22 is an isometric view of the ferrule shown in FIG. 21;

FIG. 23 is a cross-sectional view of the inventive feedthrough assembly shown in FIGS. 11, 12, and 20 deployed within an electrochemical cell;

FIG. 24 is a isometric view of an apparatus suitable for compressing and crimping the inventive feedthrough assembly;

FIG. 25 is a more detailed isometric view of the lower jaw of the apparatus illustrated in FIG. 24;

FIGS. 26 and 27 are side and isometric views of a feedthrough assembly in accordance with a second embodiment of the present invention; and

FIG. 28 is an exploded isometric view of a still further embodiment of the present invention.

### DETAILED DESCRIPTION OF THE INVENTION

The following detailed description of the invention is merely exemplary in nature and is not intended to limit the scope, applicability, or configuration of the invention in any way. Rather, the following description provides a convenient illustration for implementing exemplary embodiments of the invention. Various changes to the described embodiments may be made in the function and arrangement of the elements described herein without departing from the scope of the invention.

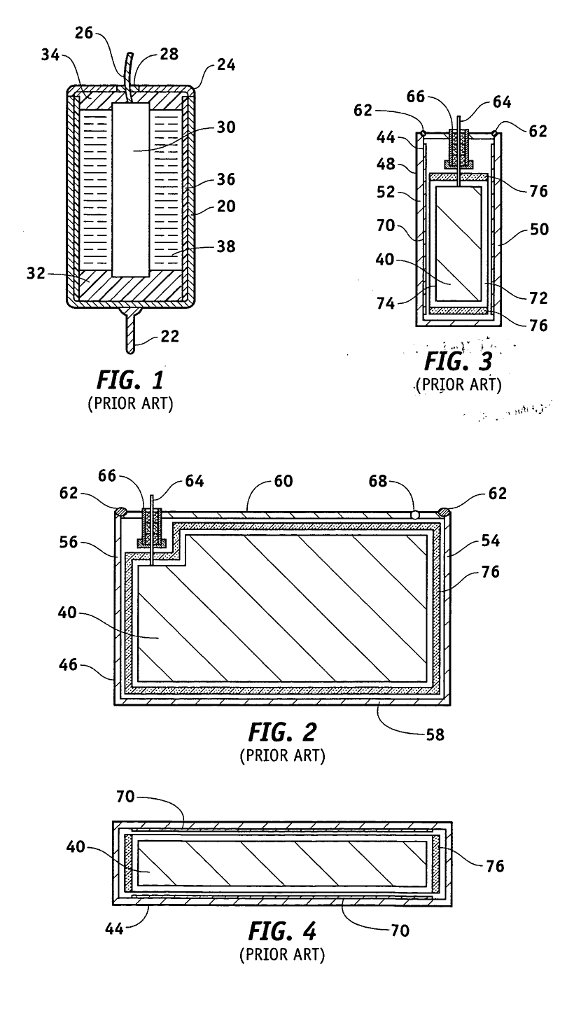

FIG. 1 is a cross-sectional view of an electrolytic capacitor in accordance with the teaching of the prior art. It comprises a cylindrical metal container 20 made of, for example tantalum. Typically, container 20 comprises the cathode of the electrolytic capacitor and includes a lead 22 that is welded to the container. An end seal of cap 24 includes a second lead 26 that is electrically insulated from the remainder of cap 24 by means of a feed-through assembly 28. Cap 24 is bonded to container 20 by, for example, welding. Feed-through 28 of lead 26 may include a glass-to-metal seal through which lead 26 passes. An anode 30 (e.g., porous sintered tantalum) is electrically connected to lead 26 and is disposed within container 20. Direct contact between container 20 and anode 30 is prevented by means of electrically insulating spacers 32 and 34 within container 20 that receive opposite ends of anode 30. A porous coating 36 is formed directly on the inner surface of container 20. Porous coating 36 may include an oxide of ruthenium, iridium, nickel, rhodium, platinum, palladium, or osmium. As stated previously, anode 30 may be made of a sintered porous tantalum. However, anode 30 may be aluminum, niobium, zirconium, or titanium. Finally, an electrolyte 38 is disposed between and in contact with both anode 30 and cathode coating 36 thus providing a current path between anode 30 and coating 36. As stated previously, while capacitors such as the one shown in FIG. 1 were generally acceptable for defibrillator applications, optimization of the device is limited by the constraints imposed by the cylindrical design.

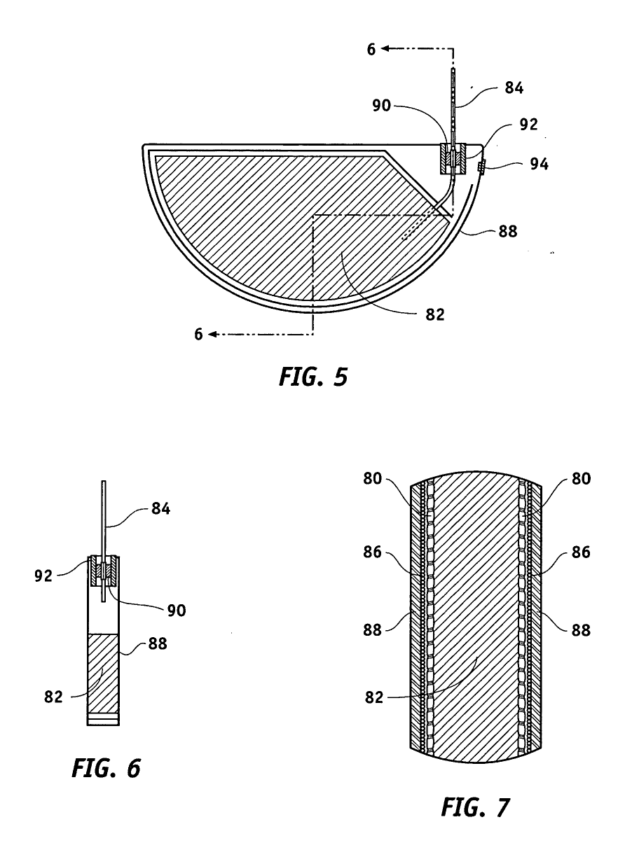

FIGS. 2, 3, and 4 are front, side, and top cross-sectional views respectively of a flat electrolytic capacitor, also in accordance with the teachings of the prior art, designed to overcome some of the disadvantages associated with the electrolytic capacitor shown in FIG. 1. The capacitor of FIGS. 2, 3, and 4 comprises an anode 40 and a cathode 44 housed inside a hermetically sealed casing 46. The capacitor electrodes are activated and operatively associated with each other by means of an electrolyte contained inside casing 46. Casing 46 includes a deep drawn can 48 having a generally rectangular shape and comprised of spaced apart side-walls 50 and 52 extending to and meeting with opposed end walls 54 and 56 extending from a bottom wall 58. A lid 60 is secured to side-walls 50 and 52 and to end walls 54 and 56 by a weld 62 to complete an enclosed casing 46. Casing 46 is made of a conductive metal and serves as one terminal or contact for making electrical connections between the capacitor and its load.

The other electrical terminal or contact is provided by a conductor or lead 64 extending from within the capacitor through casing 46 and, in particular, through lid 60. Lead 64 is insulated electrically from lid 60 by an insulator and seal structure 66. An electrolyte fill opening 68 is provided to permit the introduction of an electrolyte into the capacitor, after which opening 68 is closed. Cathode electrode 44 is spaced from the anode electrode 40 and comprises an electrode active material 70 provided on a conductive substrate. Conductive substrate 70 may be selected from the group consisting of tantalum, nickel, molybdenum, niobium, cobalt, stainless steel, tungsten, platinum, palladium, gold, silver, cooper, chromium, vanadium, aluminum, zirconium, hafnium, zinc, iron, and mixtures and alloys thereof. Anode 40 may be selected from the group consisting of tantalum, aluminum, titanium, niobium, zirconium, hafnium, tungsten, molybdenum, vanadium, silicon, germanium, and mixtures thereof. A separator structure includes spaced apart sheets 72 and 74 of insulative material (e.g. a microporous polyolefinic film). Sheets 72 and 74 are connected to a polymeric ring 76 and are disposed intermediate anode 40 and coated side-walls 50 and 52 which serve as a cathode electrode.

As already mentioned, the above described capacitors present certain concerns with respect to device size and manufacturing complexity. In contrast, FIGS. 5, 6, and 7 are front cross-sectional, side cross-sectional, and scaled cross-sectional views of an electrolytic capacitor suitable for use in an implantable medical device and utilizing a feedthrough assembly in accordance with a first embodiment of the present invention. As can be seen, one or more layers of an insulative polymer separator material 80 (e.g. micro-porous PTFE or polypropylene) are heat sealed around a thin, D-shaped anode 82 (e.g. tantalum) having an anode lead wire 84 (e.g. tantalum) embedded therein. Capacitor grade tantalum powder such as the “NH” family of powders may be employed for this purpose. These tantalum powders have a charge per gram rating of between approximately 17,000 to 23,000 microfarad-volts/gram and have been found to be well suited for implantable cardiac device capacitor applications. Tantalum powders of this type are commercially available from HC Starck, Inc. located in Newton, Mass. Of course, materials having higher or lower charge may be utilized depending upon desired results.

Before pressing, the tantalum powder is typically, but not necessarily, mixed with approximately 0 to 5 percent of a binder such as ammonium carbonate. This and other binders are used to facilitate metal particle adhesion and die lubrication during anode pressing. The powder and binder mixture are dispended into a die cavity and are pressed to a density of approximately 4 grams per cubic centimeter to approximately 8 grams per cubic centimeter. After pressing, it is sometimes beneficial to modify anode porosity to improve conductivity within the internal portions of the anode. Porosity modification has been shown to significantly reduce resistance. Macroscopic channels are incorporated into the body of the anodes to accomplish this. Binder is then removed from the anodes either by washing in warm deionized water or by heating at a temperature sufficient to decompose the binder. Complete binder removal is desirable since residuals may result in high leakage current. Washed anodes are then vacuum sintered at between approximately 1,350 degrees centigrade and approximately 1,600 degrees centigrade to permanently bond the metal anode particles.

An oxide is formed on the surface of the sintered anode by immersing the anode in an electrolyte and applying a current. The electrolyte includes constituents such as water and phosphoric acid and perhaps other organic solvents. The application of current drives the formation of an oxide film that is proportional in thickness to the targeted forming voltage. A pulsed formation process may be used wherein current is cyclically applied and removed to allow diffusion of heated electrolyte from the internal pores of the anode plugs. Intermediate washing and annealing steps may be performed to facilitate the formation of a stable, defect free, oxide.

Layers of cathode material 86 are deposited on the inside walls of a thin, shallow drawn, D-shaped casing 88 (e.g. titanium) having first and second major sides and a peripheral wall, each of which have an interior surface. In order to optimize the energy density of the electrolytic capacitor, the cathode capacitance must be several orders of magnitude higher than that of anode 82. In the past, this was accomplished by incorporating thin, etched aluminum foils between many anode layers, thus providing a large planar surface area and high capacitance. However, in order to promote downsizing as described above, the present invention employs materials of a high specific capacitance rather than large planar area. The capacitive materials may be selected from those described above or selected from the group including graphitic or glassy carbon deposited on titanium carbide, silver vanadium oxide, crystalline manganese dioxide, platinum or ruthenium on surface modified titanium, barium titanate or other perovskites on surface modified titanium, crystalline ruthenium or iridium oxide, or the like.

Anode 88 and cathode material 86 are insulated from each other by means of a micro-porous polymer separator material such as a PTFE separator of the type produced by W. L. Gore, Inc. located in Elkton, Md. or polypropylene of the type produced by Celgard, Inc. located in Charlotte, N.C. Separators 80 prevent physical contact and shorting and also provide for ionic conduction. The material may be loosely placed between the electrodes or can be sealed around the anode and/or cathode. Common sealing methods include heat sealing, ultra sonic bonding, pressure bonding, etc.

The electrodes are housed in a shallow drawn, typically D-shaped case 88 (e.g. titanium) that may have a material thickness of approximately 0.005 to 0.016 inches. An insulating feed-through 90 (to be more fully described hereinbelow) is comprised of a ferrule 92 (e.g. titanium) bonded (as, for example, by welding to case 88) to case 88. Sealed anode 82 is inserted into the cathode coated case 88, and anode lead wire 84 passes through feedthrough 90 as is shown. A lid is then positioned and secured to the case by welding.

After assembly and welding, an electrolyte is introduced into the casing through a fill-port 94. The electrolyte is a conductive liquid having a high breakdown voltage that is typically comprised of water, organic solvents, and weak acids or of water, organic solvents, and sulfuric acid. Filling is accomplished by placing the capacitor in a vacuum chamber such that fill-port 94 extends into a reservoir of electrolyte. When the chamber is evacuated, pressure is reduced inside the capacitor. When the vacuum is released, pressure inside the capacitor re-equilibrates, and electrolyte is pushed through fill-port 94 into the capacitor.

Filled capacitors are aged to form an oxide on the anode leads and other areas of the anode. Aging, as with formation, is accomplished by applying a current to the capacitor. This current drives the formation of an oxide film that is proportional in thickness to the targeted aging voltage. Capacitors are typically aged approximately at or above their working voltage, and are held at this voltage until leakage current reaches a stable, low value. Upon completion of aging, capacitors are re-filled to replenish lost electrolyte, and the fill-port 94 is sealed as, for example, by laser welding a closing button or cap over the encasement opening.

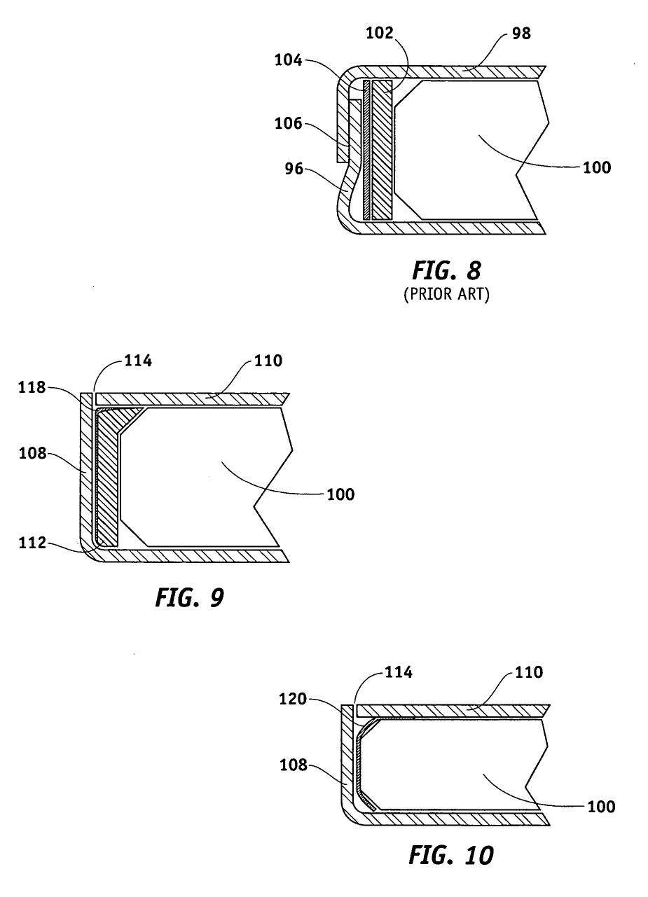

The outer metal encasement structure of a known planar capacitor generally comprises two symmetrical half shells that overlap and are then welded along their perimeter seam to form a hermetic seal. Such a device is shown in FIG. 8. That is, the encasement comprises a case 96 and an overlapping cover 98. A separator sealed anode 100 is placed within case 96, and a polymer spacer ring 102 is positioned around the periphery of anode assembly 100. Likewise, a metal weld ring 104; is positioned around the periphery of spacer ring 102 proximate the overlapping portion 106 of case 96 and cover 98. The overlapping portions of case 96 and cover 98 are then welded along the perimeter seam to form a hermetic seal.

This technique presents certain concerns relating to both device size and manufacturing complexity. The use of overlapping half-shields results in a doubling of the encasement thickness around the perimeter of the capacitor thus reducing the available interior space for the anode. Thus, for a given size anode, the resulting capacitor is larger. Furthermore, space for anode material is reduced due to the presence of weld ring 104 and insulative polymer spacer ring 102. This device is more complex to manufacture and therefore more costly.

FIG. 9 is a cross-sectional view illustrating one of the novel aspects of the present invention. In this embodiment, the encasement is comprised of a shallow drawn case 108 and a cover or lid 110. This shallow drawn encasement design uses a top down welding approach. Material thickness is not doubled in the area of the weld seam as was the situation in connection with the device shown in FIG. 8 thus resulting in additional space for anode material.

Cover 110 is sized to fit into the open side of shallow drawn metal case 108. This results in a gap (e.g. from 0 to approximately 0.002 inches) in the encasement between case 108 and cover 110 that could lead to the penetration of the weld laser beam thus potentially damaging the capacitor's internal components. To prevent this, a metallized polymeric weld ring is placed or positioned around the periphery of anode 100. Weld ring 112 is somewhat thicker than the case-to-cover gap 114 to maximize protection. Metallized weld ring 112 may comprise a polymer spacer 116 having a metallized surface 118 as shown and provides for both laser beam shielding and anode immobilization. The metallized polymer spacer 112 need only be thick enough to provide a barrier to penetration of the laser beam and is sacrificial in nature. This non-active component substantially reduces damage to the active structures on the capacitor.

Metallized polymer spacer 112 is placed around the perimeter of anode 100 during assembly and may be produced my means of injection molding, thermal forming, tube extrusion, die cutting of extruded or cast films, etc. Spacer 112 may be provided through the use of a pre-metallized polymer film. Alternatively, the metal may be deposited during a separate process after insulator production. Suitable metallization materials include aluminum, titanium, etc. and mixtures and alloys thereof.

FIG. 10 is a cross-sectional view illustrating an alternative to the embodiment shown in FIG. 9. Again, the encasement comprises a case 108 and a cover or lid 110 resulting in gap 114. The anode assembly 100 is positioned within the encasement as was the situation in FIG. 9. To protect the capacitor's internal components from damage due to the weld laser beam, a metallized tape 120 is positioned around the perimeter of anode 100.

The embodiments shown in FIGS. 9 and 10 not only have space saving aspects in the encasement design, but the components are simple and inexpensive to produce. The top down assembly facilitates fabrication and welding processes. The thinness of the weld ring/spacer 112 or metallized tape 120 reduces the need for additional space around the perimeter of the capacitor thus improving energy density. The design lends itself to mass production methods and reduces costs, component count, and manufacturing complexity.

It is not uncommon for the encasement of the capacitor itself to serve as the cathode electrode. This may be accomplished by depositing cathode material on an inner wall of the encasement or, if cathode material is deposited on one or more substrates, by electrically connecting the substrates to the encasement. Alternatively, the encasement may be made electrically neutral by not coupling the cathode to the encasement. In this situation, however, it is necessary not only to provide access to an; anode electrode at the exterior of encasement 88, but provisions must also be made to access a cathode electrode from the exterior of the encasement.

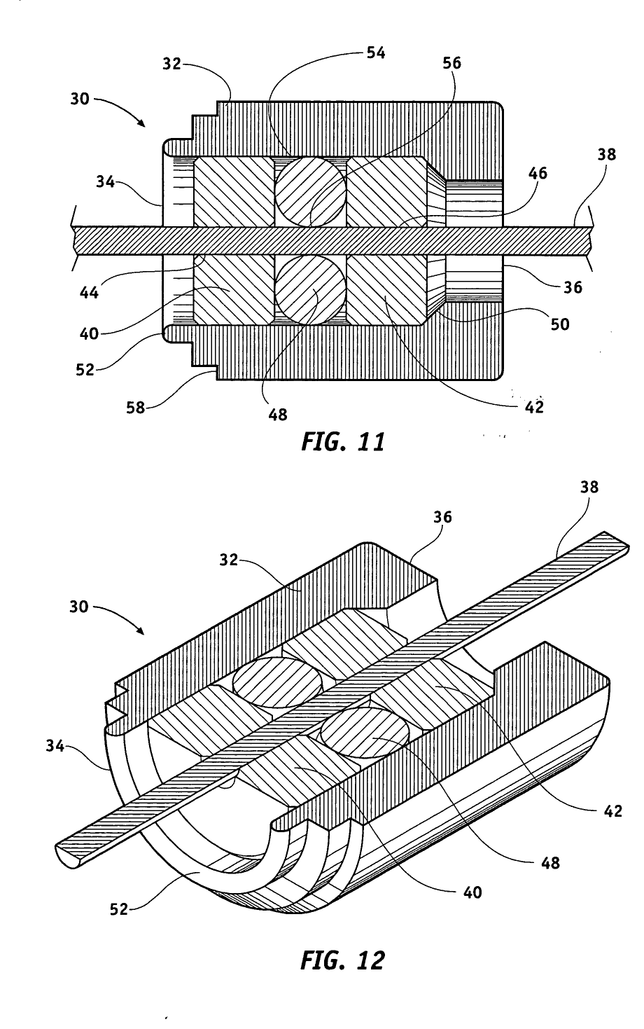

FIGS. 11 and 12 are cross-sectional and cutaway isometric views of a feedthrough assembly 30 in accordance with a first embodiment of the present invention and of the type referred to above in connection with FIGS. 5 and 6. Feedthrough assembly 30 comprises a ferrule 32 (e.g. generally cylindrical) having first and second ends 34 and 36 respectively. As can be seen, ferrule 32 is provided with openings at ends 34 and 36 so as to permit an electrical lead 38 to pass therethorugh. Positioned within ferrule 32 are first and second insulating members 40 and 42 respectively that may take the form of rings, beads, or the like. Insulating rings 40 and 42 each have an aperture therethrough (44 and 46 respectively) for receiving lead 38 therethrough. In this manner, lead 38 is guided through ferrule 32 and electrically insulated therefrom. A sealing member 48 (e.g. a compression ring) is positioned intermediate insulating rings 40 and 42 and likewise is provided with an aperture for receiving electrical lead 38 therethrough.

It should be apparent from FIGS. 11 and 12 that first end 34 of ferrule 32 is provided with an inner diameter which is larger than the inner diameter of the opening at the second end of ferrule 32 as is shown at 50. The outer diameters of insulating rings 40 and 42 and compression ring 48 are chosen to be slightly smaller than the larger inner diameter of ferrule 32 while at the same time being larger than the inner diameter of ferrule 32 at second end 36. In this way, insulating rings 40 and 42 and compression ring 48 may be threaded around lead 38, and the entire assembly slipped through opening 34 and positioned within ferrule 32. This retaining portion (i.e. the reduced inner diameter of ferrule 32 shown at 50) prevents the insulating ring/compression ring assembly from being pushed through the second opening in ferrule 32.

The first end of ferrule 32 is also provided with a retaining portion 52 (e.g. a circular collar or one or more tabs) which is ultimately crimped or compressed as will be described hereinbelow so as to compress the insulating ring/compression ring assembly. This configuration is illustrated in a cross-sectional view, shown in FIG. 20 wherein like elements are denoted with like reference numerals. It can be seen that collar 52 has been crimped in a way so as to compress ring 48 between insulating rings 40 and 42 thus deforming compression ring 48 and causing it to sealingly engage the exterior surface of lead 38 as is shown at 56 and the interior surface of ferrule 22 as is shown at 54. Finally, referring again to FIGS. 11 and 12, first end 34 of ferrule 32 is provided with a stepped portion or receiving shoulder 58 which will facilitate the mounting of feedthrough 32 within the wall of an electrochemical cell as will be more fully described below.

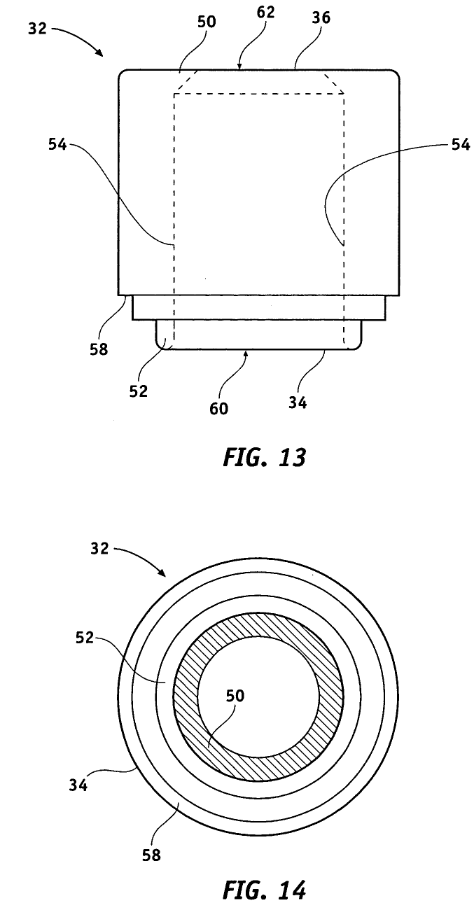

FIGS. 13, 14, and 15 are front, bottom, and isometric views of ferrule 32 utilized in conjunction with the inventive feedthrough shown in FIGS. 11, 12, and 20. As stated previously, ferrule 32 may be generally cylindrical in shape; however, it should be understood that ferrule 32 may take any desired shape to suit a particular application. As can be seen, ferrule 32 includes a first opening 60 having a first inner diameter and a second opening 62 having an inner diameter smaller than the inner diameter of opening 60 for the reasons described above. For example, opening 60 may have an inner diameter of approximately 0.057 inch whereas opening 62 may have an inner diameter of 0.05 inch. The outer diameter of ferrule 32 may be approximately 0.09 inch, and ferrule 32 may have a length of approximately 0.08 inch. Obviously, these dimensions may be varied to suit a particular application. Ferrule 32 may be made of titanium having a grade 1-5, preferably 2. Other suitable metals such as niobium, stainless steel, aluminum, copper, etc., may be utilized.

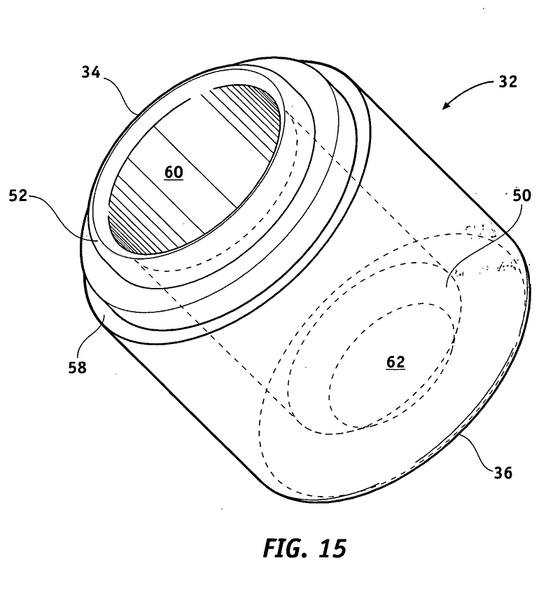

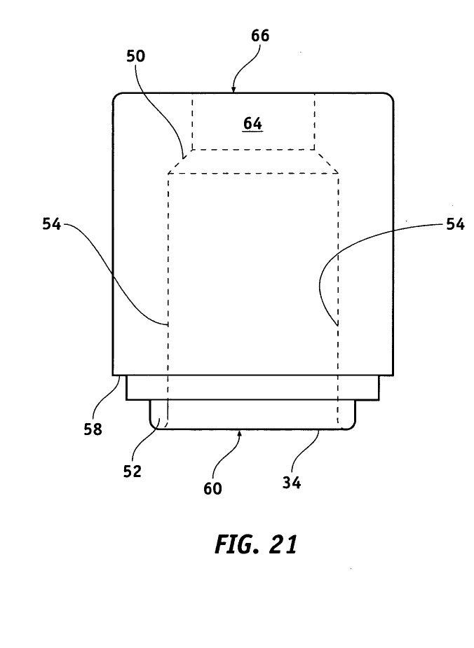

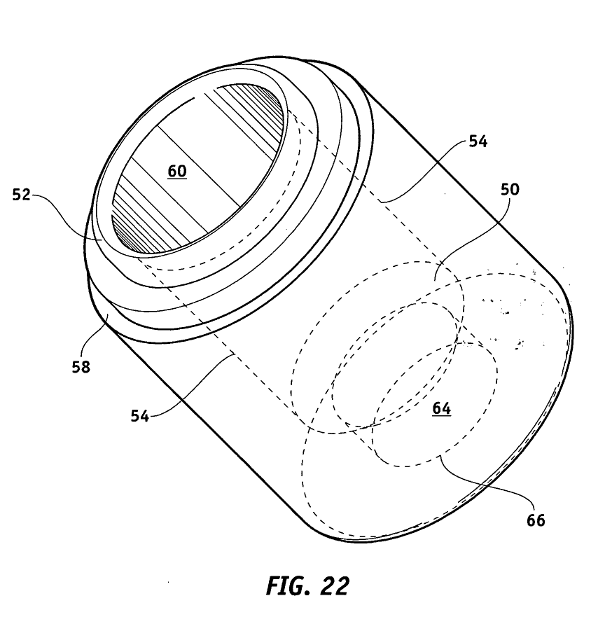

FIGS. 21 and 22 illustrate a second embodiment of a ferrule suitable for use in conjunction with the inventive feedthrough assembly. Again like elements are denoted by like reference numbers. Referring to FIG. 21, it can be seen that the length of ferrule 32 has been extended to include a hollow tubular extension 64 having opening 66 at one end thereof. Extension 64 has an inner diameter which may be substantially equal to the inner diameter of opening 62 shown in FIG. 15; however, extension 64 may have any desired inner diameter or length so long as an area of reduced diameter such as is shown at 50 is provided to abuttingly engage insulating ring 42 to maintain insulating ring 42 in position.

An electrical lead (not shown in FIG. 21) passes through the insulating ring/compression ring assembly as previously described and continues out through extension 64. The lead may then be fixtured by any suitable means outside the feedthrough assembly. Once fixtured, a cavity within extension 64 may be filled with a suitable insulating material such as epoxy. The epoxy, in addition to insulating the electrical lead from the ferrule, also provides addition sealing. Furthermore, the epoxy provides strain relief by protecting the lead from mechanical stresses. That is, the epoxy fixedly positions the lead with respect to the ferrule and absorbs mechanical stresses placed on the lead.

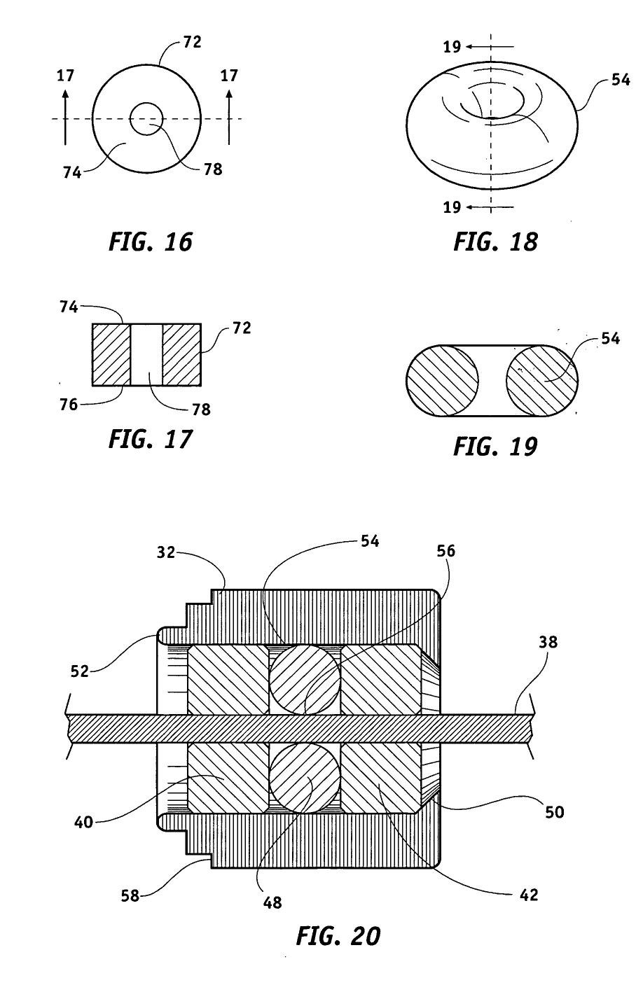

FIGS. 16 and 17 are top and cross-sectional views of an insulating ring suitable for use in conjunction with the invention feedthrough assembly. As can be seen, the insulating ring is generally cylindrical in shape having a substantially cylindrical outer wall 72, substantially flat upper and lower surfaces 74 and 76 respectively, and an aperture 78 through which lead 38 (FIG. 11) may pass. For example, insulating ring 72 may have an outer diameter of approximately 0.055 inch, and aperture 78 may have diameter of 0.017 inch and a height of approximately 0.030 inch. In a preferred embodiment, the insulating ring is made of chromium doped alumina which is extremely strong so as to withstand the compression forces described above without damage. Other materials may be suitable for this purpose including hard plastic, glass, alumina, fired insulators such as porcelain, etc. The specific material used will be determined by the specific application and the device chemistry. It is only necessary that the material be insulative and harder than the material from which compression ring 48 is manufactured.

FIGS. 18 and 19 are isometric and cross-sectional views of a compression ring suitable for use in the inventive feedthrough assembly shown in FIGS. 11, 12, and 20. Compression ring 54 is preferably made of a silicon based material; however, other substances may be utilized such as a flouroelastomer co-polymer of vinylidene fluoride and hexaflouropropylene, ethylene propylene diene monomer rubber, polychloroprene, acrylonitrile butadiene co-polymer, polysulphide, etc., depending on the particular application and device chemistry. An as example, compression ring 54 may have an outer diameter of 0.056 inch and a height of 0.02 inch. The particular compression ring shown in FIGS. 18 and 19 is shown as having a circular cross-section; however, it should be appreciated that compression ring 54 may take any desired shape and could if desired have a cross-section that is square, rectangular, or other suitable shape.

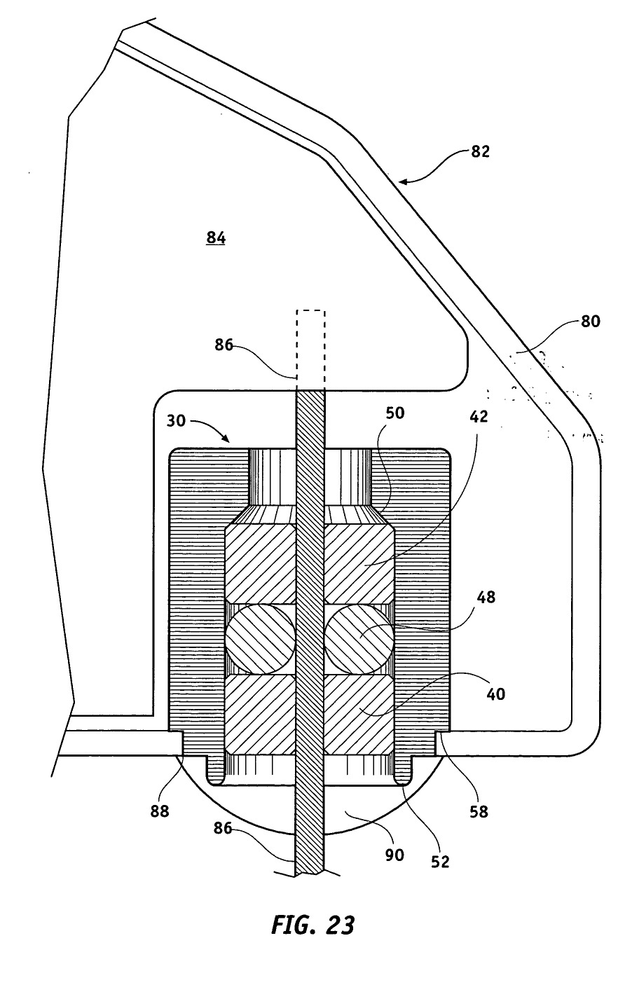

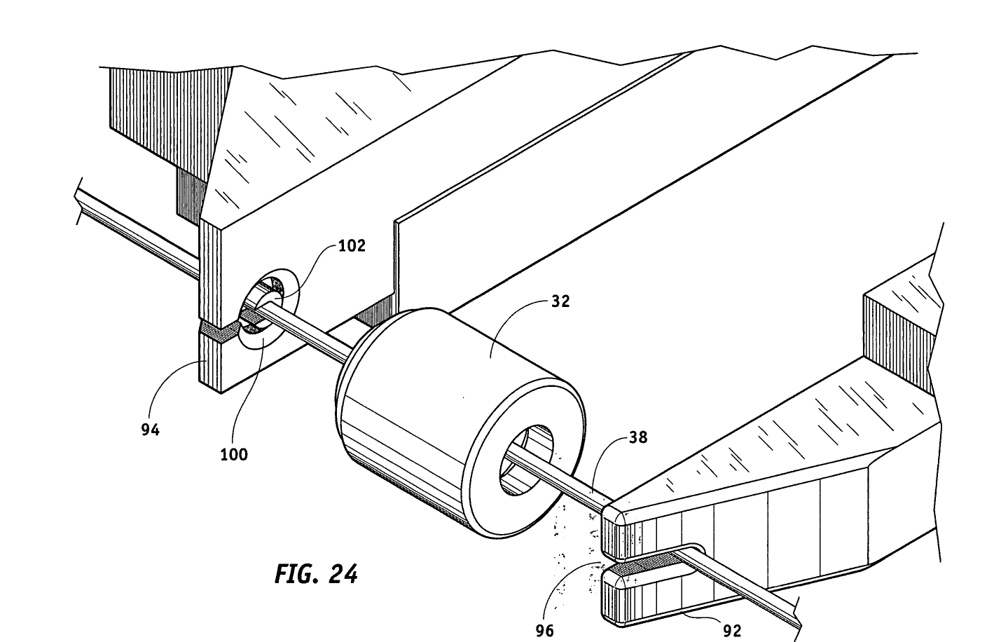

FIG. 23 is a cross-sectional view illustrating the deployment of the inventive feedthrough assembly shown in FIGS. 11, 12, and 20 within an encasement 80 of an electrochemical cell 82 such as a capacitor, battery, sensor, or the like. As can be seen, an anode 84 having an anode lead 86 embedded therein is positioned within encasement 80. Anode lead 86 passes through feedthrough assembly 30 as described above. In this manner, contact may be made to anode lead 86 from the exterior of encasement 80.

As was referred to earlier, feedthrough assembly 30 is provided with a receiving shoulder 58 shown in FIGS. 11 and 23. This receiving shoulder is placed in an abutting relationship with the edges of an aperture in encasement 80 resulting in the production of a circular seam at the exterior of encasement 80 which facilitates the process of laser welding ferrule 32 to encasement 80. After the ferrule is fixedly coupled to encasement 80, anode lead 86 is passed through the ferrule to the exterior of the, encasement. Insulating ring 42 is then threaded onto lead 86 followed by compression ring 48 and insulating ring 40. As can be seen, insulating ring 42 abuttingly engages region 50 of reduced inner diameter.

After proper positioning of the insulator rings and compression ring within ferrule 32, collar 52 is crimped over the edge of insulating ring 40. This is accomplished by means of a compression process which not only causes collar 52 to be crimped over the edge of insulating ring 40, but also compresses the insulating ring/compression ring stack so as to deform compression ring 48 causing it to sealingly engage the inner surface of the ferrule and anode lead 86. In this manner, the sealing and insulating components (i.e. insulating rings 40 and 42 and compression ring 48) are not subjected to thermal stresses during the welding process since they are not inserted into the ferrule until after the ferrule has been welded to encasement 80.

If desired, a body of insulating material such as epoxy may be deposited around anode lead 86 and the exposed portion of the feedthrough assembly as is shown in FIG. 23. This body of epoxy protects anode lead 86 from mechanical stresses and positions the anode lead with respect to the feedthrough and encasement. Thus, epoxy 90 absorbs some of the mechanical stresses to which anode 86 may be exposed and also functions as a redundant seal.

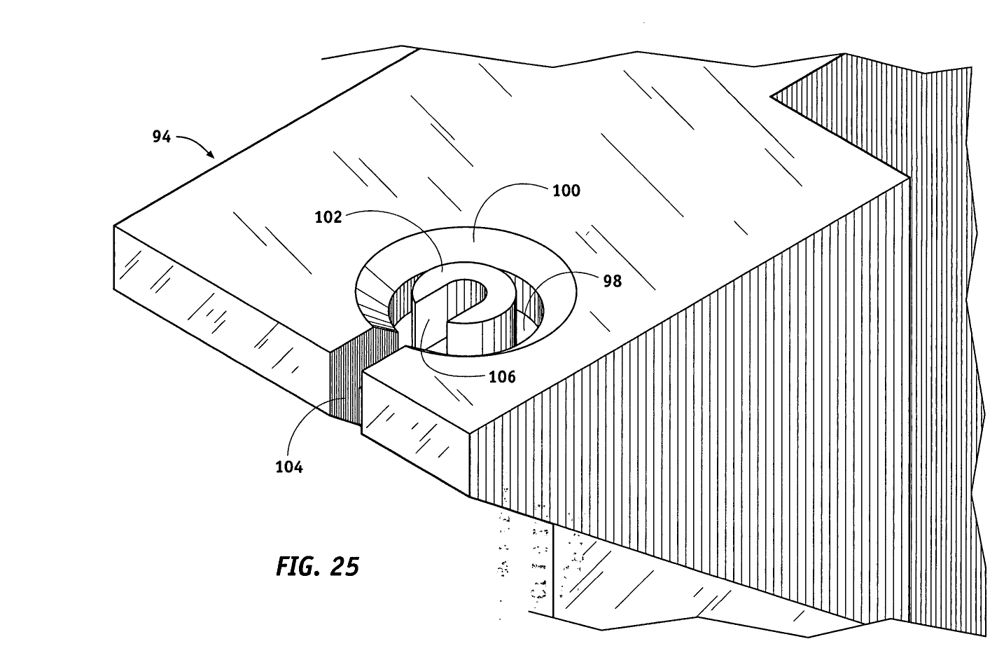

FIG. 24 illustrates an apparatus for performing the required compression and crimping as described above. As can be seen, the apparatus comprise a first compression jaw 92 and a second compression jaw 94 shown in more detail in FIG. 25. It should be understood that the compression process takes place after ferrule 32 has been fixedly attached to encasement 80 as shown in FIG. 23; however, for simplicity, encasement 80 is not shown in FIG. 24.

Compression jaw 92 includes an opening 96 such as a slot, for example, for receiving a first end portion of lead 38. Compression jaw 94 includes a well 98 having an inclined surface 100. Protruding upward from a central portion of well 98 is an island 102. Jaw 94 and island 102 likewise contain openings (for example in the form of slots) 104 and 106 respectively for receiving another portion of anode lead 38 as is shown in FIG. 24. Once properly positioned, jaws 92 and 94 compressingly engage feedthrough assembly 30. During this process, island 102 engages insulating ring 40 so as to compress the insulation ring/compression ring assembly causing compression ring 48 to deform and sealingly engage both the inner surface of ferrule 32 and the surface of anode lead 38. Substantially simultaneously therewith, inclined surface 100 bears against collar 52 causing it to bend over the edge of insulating ring 40. The pressure exerted by compression jaws 92 and 94 is chosen so as to achieve the desired compression and crimping without damaging compression ring 48. Typically compression jaws 92 and 94 will exert a force in the range of 0.5-10. It should be understood that compression jaws 92 and 94 may form a part of an automated system. In contrast, however, jaws 92 and 94 may simply comprise the jaws of a special purpose compression tool similar too well-known hand pliers. Since the compression process is performed prior to welding the encasement lid to the case, it is a relatively simple matter to insert jaw 92 into the case between anode 84 (FIG. 23) and ferrule 32.

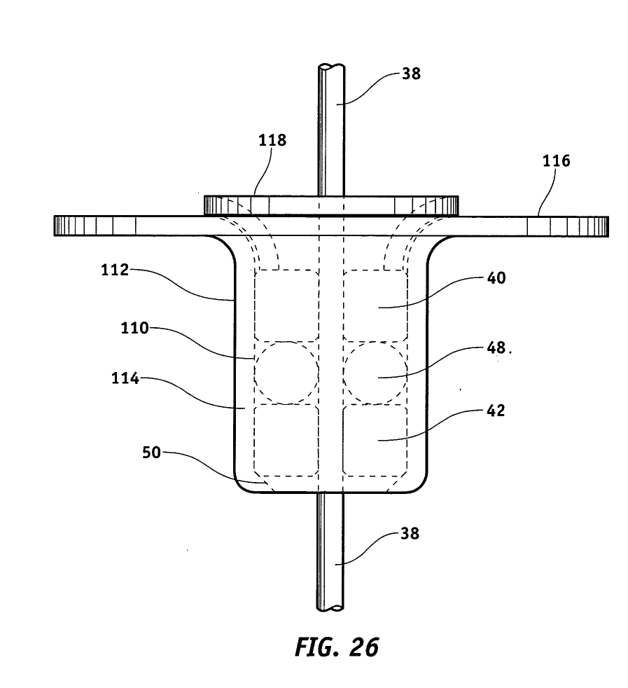

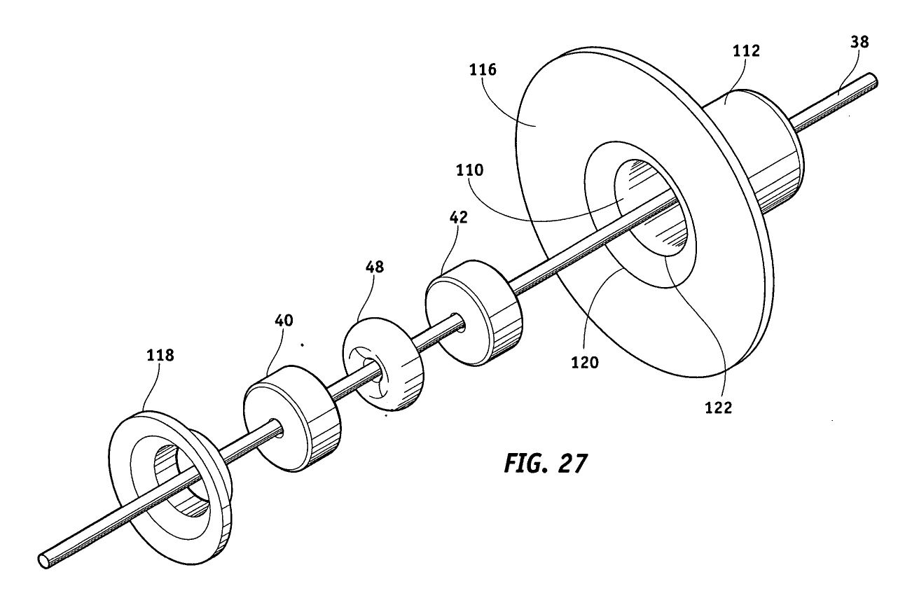

FIGS. 26 and 27 are side and isometric views of a feedthrough assembly in accordance with a second embodiment of the present invention. Like elements are denoted by like reference numerals. As was the case previously, a compression ring 48 is compressed between first and second insulating rings 40-and 42 respectively and sealingly engages the surface of lead 38 and the inner surface 110 of ferrule 112. Ferrule 112 has a substantially cylindrical portion 114 having a first end of reduced inner diameter 50 for the reasons described above in connection with FIGS. 11 and 13-15. As was the case previously, the compression ring/insulating ring assembly is positioned within the cylindrical portion 114 of ferrule 112, and lead 38 passes through the compression ring/insulating ring assembly and therefore through ferrule 112 itself. The second or opposite end of ferrule 112 terminates in a mounting plate. 116 that flairs outwardly from the wall of cylindrical portion 114 forming a ferrule entrance; 120 having a generally tapered or curved surface 122. This plate may be coupled (e.g. welded) around an aperture in the encasement wall of an electrochemical cell (not shown).

A collar 118 having an aperture therethrough for receiving lead 38 is shaped so as to be generally matingly received within entrance 120 as is shown in FIG. 26, and in this manner compresses the compression ring/insulating ring assembly for the reasons described above. Compression collar 118 may then be fixedly coupled (e.g. by spot welding at least one location) to mounting plate 116.

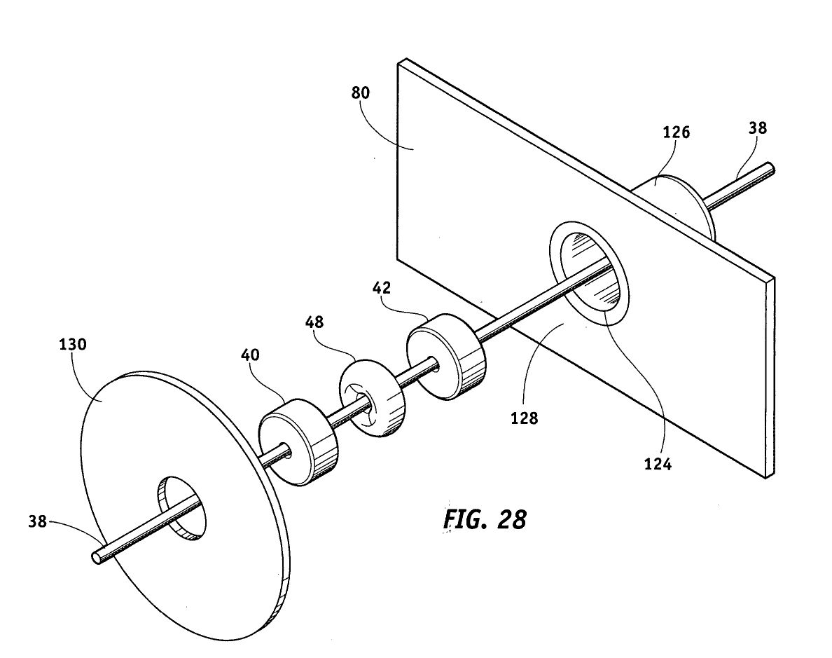

FIG. 28 is an exploded view of a feedthrough assembly similar to that shown and described in connection with FIGS. 26 and 27 except that a first end 124 of a substantially cylindrical ferrule 126 is fixedly coupled (as by laser welding) around an opening in the encasement wall 128 of an electrochemical cell (not shown) and collar 118 (FIG. 27) is replaced by a compression plate 130 having an aperture therethrough for receiving lead 38. Compression plate 130 is fixedly coupled (as by laser welding) to encasement wall 128 thus compressing the compression ring/insulating ring assembly within ferrule 126 to create the necessary seal between compression ring 48, lead 38, and the inner surface of ferrule 126.

Thus, there has been provided a miniature compression feedthrough assembly for use in electrochemical cells such as capacitors, batteries, and the like for use in implantable medical devices. The inventive feedthrough assembly provides for a polymer-to-metal seal which offers greater chemical stability over conventional glass-to-metal seals in certain chemical electrolyte environments. The inventive design simplifies assembly by eliminating the need for internal cross-wire welds and improves volumetric efficiency by eliminating significant headspace volume in the capacitor. The inventive feedthrough assembly can be entirely assembled from outside the cell thus avoiding thermal stresses on critical feedthrough components such as the compression ring during welding. While the invention has been described in conjunction with a feedthrough assembly incorporating first and second insulating rings and a single compression rings, it should be appreciated that any number of insulating rings and compression rings could be utilized to satisfy the requirement of a given application.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A miniature insulative feedthrough for receiving an electrical lead therethrough, said feedthrough comprising;
a ferrule having first and second open ends and an interior surface;
at least a first insulating ring positioned within said ferrule and having an aperture therethrough for receiving said electrical lead;
at least one compression ring positioned within said ferrule for sealingly engaging said interior surface, said at least one compression ring having an aperture therethrough for receiving said electrical lead; and
first and second retaining portions for maintaining said at least one insulating ring and said at least one compression ring in position within said ferrule.

2. A miniature insulative feedthrough according to claim 1 further comprising a second insulating ring positioned within said ferrule and having an aperture therethrough for receiving said electrical lead, said at least one compression ring positioned intermediate said first and second insulating rings for compression therebetween.

3. A miniature insulative feedthrough according to claim 2 wherein said first retaining portion is a crimped portion of said ferrule.

4. A miniature insulative feedthrough according to claim 3 wherein said second retaining portion is a first region of reduced dimensions in said ferrule.

5. A miniature insulative feedthrough according to claim 4 wherein said ferrule is generally cylindrical in shape, said crimped portion is a collar coupled at said first open end, and said first region has a reduced diameter proximate said second open end.

6. A miniature insulative feedthrough according to claim 5 wherein said ferrule further comprises a generally tubular extension coupled to said second open end.

7. A miniature insulative feedthrough according to claim 6 wherein said tubular extension contains an insulating material.

8. A miniature insulative feedthrough according to claim 7 wherein said insulating material is epoxy.

9. A miniature insulative feedthrough according to claim 4 wherein said at least one compression ring is made of a polymeric material.

10. A miniature insulative feedthrough according to claim 9 wherein said at least one compression ring has a substantially circular cross-section prior to compression between said at least first and second insulating rings.

11. A miniature insulative feedthrough according to claim 10 wherein said first and second insulating rings are alumina.

12. A miniature insulative feedthrough according to claim 4 wherein said ferrule is made of titanium.

13. A miniature insulative feedthrough according to claim 10 wherein said first and second insulating rings have a hardness greater than that of said compression ring.

14. A method for feeding a terminal lead through an encasement wall of an electrochemical device of the type utilized in implantable medical devices, of the method comprising:
positioning a ferrule having first and second ends through said encasement wall, said ferrule having an interior surface and having a crimping region at said first end;
passing said terminal lead through said ferrule;
threading said terminal lead through an assembly comprised of at least first and second insulating rings and at least one intermediate compression ring;
positioning said assembly within said ferrule; and
compressing said crimping portion and said assembly to maintain said assembly within said ferrule and to deform said compression ring to sealingly engage said interior surface.

15. A method according to claim 14 wherein positioning said ferrule comprises welding said ferrule to said encasement along an exterior seam between said second end and an exterior surface of said encasement, said first end remaining inside said encasement.

16. A method according to claim 15 wherein said terminal lead is passed through said ferrule from said first end to said second end.

17. A method according to claim 16 wherein said encasement is a shallow drawn encasement comprising a case having a major side and a peripheral wall, and a lid for coupling to said peripheral wall, and wherein said compressing takes place before said lid is coupled to said wall.

18. A method according to claim 16 wherein said crimping portion is a generally cylindrical collar and said compressing includes collapsing said collar inward.

19. A method according to claim 15 wherein said ferrule is welded to said encasement prior to positioning said assembly within said ferrule.

20. A method according to claim 19 wherein said ferrule is welded to said encasement prior to said threading.

21. An electrochemical cell for use in an implantable medical device, said electrochemical cell comprising:
an encasement;
at least one electrode body disposed within said encasement;
an electrical lead coupled to said electrode body; and
an insulative feedthrough coupled through said encasement for receiving said electrical lead therethrough, said insulative feedthrough comprising:
a ferrule having first and second open ends and an interior surface;
at least first and second insulating rings positioned within said ferrule and each having an aperture therethrough for receiving said electrical lead;
at least one compression ring positioned within said ferrule and compressed between said at least first and second insulating rings so as to sealingly engage said interior surface, said at least one compression ring having an aperture therethrough for receiving said electrical lead; and
first and second retaining portions for maintaining said at least first and second insulating rings in position within said ferrule.

22. An electrochemical cell according to claim 21 wherein said first retaining portion is a crimped portion of said ferrule.

23. An electrochemical cell according to claim 21 wherein said first retaining portion comprises a compression collar inserted into said first open end.

24. An electrochemical cell according to claim 21 wherein said first retaining portion comprises a compression plate fixedly coupled to said encasement.

25. An electrochemical cell according to claim 22 wherein said second retaining portion is a first region of reduced dimensions in said ferrule.

26. An electrochemical cell according to claim 25 wherein said ferrule is generally cylindrical in shape, said crimped portion is a collar coupled at said first open end, and said first region has a reduced diameter proximate said second open end.

27. An electrochemical cell according to claim 26 wherein said ferrule further comprises a generally tubular extension coupled to said second open end.

28. An electrochemical cell according to claim 27 wherein said tubular extension contains an insulating material.

29. An electrochemical cell according to claim 28 wherein said insulating material is epoxy.

30. An electrochemical cell according to claim 25 wherein said at least one compression ring is made of a polymeric material.

31. An electrochemical cell according to claim 30 wherein said at least one compression ring has a substantially circular cross-section prior to compression between said at least first and second insulating rings.

32. An electrochemical cell according to claim 31 wherein said first and second insulating rings are alumina.

33. An electrochemical cell according to claim 25 wherein said ferrule is made of titanium.

34. An electrochemical cell according to claim 31 wherein said first and second insulating rings have a hardness greater than said compression ring.

35. An electrochemical cell according to claim 21 wherein said electrochemical cell is a capacitor.

36. An electrochemical cell according to claim 35 wherein said encasement comprises a case having first and second major sides and a peripheral wall coupled to said first and second major sides.

37. An electrochemical cell according to claim 36 wherein said at least one electrode body comprises:
a cathode disposed within said encasement proximate said first and second major sides; and
a centrally disposed anode within said encasement, said anode having an anode lead.

38. An electrochemical cell according to claim 37 wherein said capacitor structure comprises an electrolyte within said encasement and in contact with said cathode and said anode.

39. An electrochemical cell according to claim 38 wherein said capacitor structure further comprises a first insulative separator between said anode and said cathode.

40. An electrochemical cell according to claim 39 wherein said capacitor structure further comprises a second insulative separator between said cathode and said first and second major sides.

41. An electrochemical cell according to claim 40 wherein said capacitor structure further comprises at least one substrate having cathode material deposited thereon.

42. An electrochemical cell according to claim 37 wherein said encasement further comprises;
said first major side and said first peripheral wall; and
a lid including a second major side and sealingly coupled to said case along adjacent edges of said lid and said wall.

43. An electrochemical cell according to claim 42 further comprising a protective layer on said anode adjacent said peripheral wall to protect said at least one of said first and second anodes when said lid is sealing coupled to said case.

44. An electrochemical cell according to claim 43 wherein said protective layer comprises a metallized ring.

45. An electrochemical cell according to claim 44 wherein said metallized ring comprises a polymer spacer having a metallized surface.

46. An electrochemical cell according to claim 43 wherein said protective layer comprises a metallized tape.

47. A compression apparatus for producing a seal between a compression ring and an inner surface of a ferrule having a crimping portion at a first end thereof and having a second end, an insulating member being positioned between said first end and said compression ring, and an electrical lead passing through said ferrule, said compression apparatus comprising:
a first jaw member for compressingly engaging said second end; and
a second jaw member for compressingly engaging said insulating member and said crimping portion to compress said compression ring and deform said crimping portion.

48. A compression apparatus according to claim 47 wherein said first jaw member includes a first opening for receiving said lead proximate said second end.

49. A compression apparatus according to claim 48 wherein said second jaw member comprises:
a well having an inclined surface for forcing said crimping portion against said insulating member;
an island protruding from a central portion of said well for engaging said insulating member; and
a second opening extending into said well and said island for receiving said lead proximate said first end.

50. A compression apparatus according to claim 49 wherein said first opening is a slot.

51. A compression apparatus according to claim 50 wherein said second opening is a slot extending to a central portion of said well and said island.
